(19) United States
(12) Patent Application Publication
(10) Pub. No.: US 2023/0160818 A1
(43) Pub. Date: May 25, 2023

(54) SYSTEMS AND METHODS FOR PREDICTING A RISK OF DEVELOPMENT OF BRONCHOPULMONARY DYSPLASIA

(71) Applicant: Sime Diagnostics Ltd., London (GB)

(72) Inventor: Henrik Verder, Bagsværd (DK)

(21) Appl. No.: 17/906,800

(22) PCT Filed: Mar. 26, 2021
(86) PCT No.: PCT/EP2021/057944

§ 371 Date: Sep. 20, 2022

(30) Foreign Application Priority Data
Mar. 26, 2020 (EP) 20165923.2

(51) Int. Cl. G01N 21/3577; G06N 20/10; G01N 33/50

(52) U.S. Cl. CPC G01N 21/3577; G06N 20/10; G01N 33/50; G01N 2021/3595

(57) ABSTRACT

The present disclosure relates to a computer-implemented method for predicting a risk of an infant developing bronchopulmonary dysplasia (BPD), the method comprising the steps of: obtaining a dataset, of the infant, comprising a. clinical data; b. lung maturity data; and c. gastric aspirate (GAS) data; analysing said dataset, thereby obtaining an analysed data result; and based on said analysed data result predicting the risk of the infant developing BPD.

The present disclosure relates to a computer-implemented method, a method for supervised training of a machine learning model for predicting BPD, and a system, for predicting a risk of an infant developing bronchopulmonary dysplasia (BPD).

### BACKGROUND

Prematurely born infants, especially those born before 28 weeks of gestation, have very few alveoli at birth. The alveoli that are present tend to not be mature enough to function normal, and the infant may require respiratory support with oxygen to upkeep breathing.

Bronchopulmonary dysplasia (BPD) is typically suspected when a ventilated infant is unable to wean from prolonged high oxygen delivery. Various diagnosis criteria for BPD exist, but commonly relies on that the patient requires supplemental oxygen supply for an extended time following birth, most often 28 days. If this criterion is fulfilled, chest x-rays of the patient are typically taken and examined for signs that are characteristic for BPD, including emphysema, pulmonary scarring, and atelectasis.

While clinical classification of BPD relies on the assessment of supplemental oxygen supply at a later stage in life, typically at the 28th day of life, it is known that early treatments, including administration of steroids before the eighth day of life, can prevent development of BPD. The risk associated with said treatments may however outweigh the benefits, making treatment only a suitable option after confirmation of the disease. Thereby, there is a significant need for early prediction of development of BPD, as it can help decrease both the associated short-term and long-term effects of the disease.

### SUMMARY OF INVENTION

Early prediction of development of BPD is of paramount importance for an effective intervention of the disease. Various clinical factors and biomarkers have been investigated for the assessment of the risk of an infant developing BPD, such as clinical scoring systems, plasma proteome analyses, and blood-cell counting (neutrophil-to-lymphocyte ratio).

The present inventors have realized that development of BPD can be predicted, with high sensitivity and specificity, early after birth, by analysis of gastric aspirate (GAS) data, clinical data and lung maturity data. The early prediction of development of BPD enables the possibility of ensuring adequate treatment of the infant, and thereby, providing potential for decreasing the significant mortality and morbidity associated with the disease.

The present invention therefore, in a first aspect, relates to a computer-implemented method for predicting risk of an infant developing bronchopulmonary dysplasia (BPD), the method comprising the steps of:
a) obtaining a dataset, of the infant, comprising:
   clinical data;
   lung maturity data; and
   gastric aspirate (GAS) data;
b) analysing said dataset, thereby obtaining an analysed data result; and
c) based on said analysed data result predicting the risk of the infant developing BPD

The GAS data is preferably provided as spectroscopy data, for example mid-infrared spectroscopy data. A preferred spectrum for GAS data includes the wavelengths in the range 900-3400 cm⁻¹, such as in the range 900-1800 cm⁻¹ and in the range 2800-3400 cm⁻¹. FTIR spectral data, e.g. measurement data at spectral lines indicative of development of BPD, may be selected and form a basis, together with additional data of the dataset, for the prediction of development of BPD in the infant.

Secondly, the dataset may include clinical data comprising markers associated with development of BPD, such as gestational age and/or birth weight.

Thirdly, the dataset may further comprise lung maturity data indicative of the maturity of the lungs. Preferably, the lung maturity data is provided in the form of a binary value (+/−) of whether the infant has been given, or is to be given surfactant treatment.

Surfactant treatment (surfactant replacement therapy) may for example be given to infants with RDS in order to keep the alveoli from sticking together, and is in most cases administered in combination with supplemental oxygen or mechanical ventilation to help the infant breathe.

In a further aspect, the present invention relates to a method for supervised training of a machine learning model for predicting, early after birth, if a subject (e.g. an infant) is at risk of developing BPD. Preferably, the method comprises obtaining a dataset comprising information of a number of infants, shortly after birth. A machine learning model may thereafter be trained based on said dataset, together with outcome data comprising information related to whether said infants had, or developed, BPD. The dataset preferably comprises clinical data, lung maturity data and/or GAS data.

As shown by the present inventors, gastric aspirate of infants that develops BPD soon after birth, and gastric aspirate of infants that does not develop BPD are distinct. In fact, gastric aspirate, which is mainly produced in the foetal lungs, provides a highly detailed digital fingerprint of the foetal lung biochemistry, which may be used to predict development of BPD.

In an embodiment of the present disclosure, an artificial intelligence (AI) model is trained, based on outcome data, to select data points or spectral lines of a gastric aspirate measurement, wherein the data points or spectral lines are selected to most accurately distinguish between infants that develop BPD and those who do not develop BPD. As such, the training of the machine learning model may not require a priori knowledge of the relevant molecules and biomarkers of the gastric aspirate. The training might be supervised training of the AI model.

In yet a further aspect, the present invention relates to a system for predicting if an infant, early after birth, is at risk of developing BPD, the system comprising a memory, and a processing unit that is configured to carry out the computer-implemented method as disclosed herein. Preferably said system further comprises at least one spectrometry unit for obtaining spectrometry data, such as a spectrometer.

### DESCRIPTION OF DRAWINGS

FIG. 1 shows a flowchart of a study of development of BPD with the inclusion and number of infants with BPD and no BPD.

FIG. 2 shows the results of use of a trained machine learning model, according to an embodiment of the present disclosure, for the prediction of bronchopulmonary dysplasia based on spectral data of gastric aspirates.

FIG. 3 shows the results of use of a trained machine learning model, according to an embodiment of the present disclosure, for the prediction of bronchopulmonary dysplasia based on spectral and clinical data of gastric aspirates.

### DETAILED DESCRIPTION

In a first aspect, the present disclosure relates to a computer-implemented method for predicting a risk of an infant developing bronchopulmonary dysplasia (BPD). The method comprises the steps of: obtaining a dataset of the infant, the dataset comprising clinical data; lung maturity data; and gastric aspirate (GAS) data; analysing said dataset, thereby obtaining an analysed data result; and based on said analysed data result predicting the risk of the infant developing BPD.

In a preferred embodiment of the present disclosure, the analysed data result is obtained by analysing the dataset by a trained machine learning model. Thereby, no human intervention may be needed for carrying out the analysis, and the trained machine learning model may be continuously optimized based on new data, e.g. training data.

Preterm birth, also known as premature birth, is the birth of a baby at fewer than 37 weeks' gestational age, as opposed to the usual about 40 weeks. Thereby, in yet a preferred embodiment of the present disclosure, the infant is a preterm born infant, such as an infant born before 37 weeks of pregnancy are completed. The infant may however be born at an earlier stage of pregnancy, such as less than 35 weeks' gestational age, or even less than 30 weeks' gestational age. The risk of development of BPD is higher at a lower gestational age.

A cause to this correlation is likely the less developed lungs of an early born infant. In general, around 16-26 weeks postmenstrual age (PMA) alveoli and lung capillaries are formed. After around 26 weeks PMA, the saccules grow in size while at around week 32 the alveoli develop. Thereby, a premature birth may be associated with underdeveloped lungs, wherein a lower gestational age means less developed lungs. The incidence of BPD in surviving infants less than or equal to 28 weeks gestational age has been relatively stable at approximately 40% over the last few decades

A significant advantage with the presently disclosed method is that it enables early prediction of development of BPD. Consequently, in an embodiment of the present disclosure, the dataset comprises or consists of data obtained within 48 hours after birth, more preferably within 36 hours after birth, most preferably within 24 hours after birth, such as at birth. The earlier the data of the dataset can be obtained, the earlier a prediction of the development of BPD in an infant can be made, and consequently, the earlier a targeted intervention can be started, having the potential to significantly improve outcome. The early intervention may comprise preventative and targeted prophylactic, therapeutic intervention with surfactant and new medicaments, and/or the mode of ventilation. Various strategies for treatment and preventive therapy of BPD are known to a person skilled in the art.

GAS Data

In an embodiment of the present disclosure the GAS data, is derived from, such as comprises or consists of, spectroscopy data, for example mid-infrared spectroscopy data. The GAS data may be derived from, or comprise, spectroscopy data in the spectrum between 900-3400 cm⁻¹, such as between 900-1800 cm⁻¹ and between 2800-3400 cm⁻¹. Spectroscopy measurements of GAS, for example by FTIR spectroscopy, enable derivation of a highly detailed digital fingerprint of the foetal lung biochemistry. Thereby, GAS data may comprise FTIR spectral wavelengths and/or absorption intensities and may, combined with other markers, be evaluated for the prediction of BPD. The highly detailed digital fingerprint of the foetal lung biochemistry, is at least in part due to GAS comprising fluid that is produced in the foetal lungs.

In an embodiment of the present disclosure the GAS data is derived from, such as comprises or consists of, one or more absorption and/or one or more transmission spectra. The GAS data may consist of data derived from a single spectroscopy measurement, or the GAS data may comprise data derived from multiple spectroscopy measurements. Furthermore, the multiple measurements may have been carried out on different types of bodily fluids. In a preferred embodiment of the present disclosure the GAS data is derived from measurements of a GAS sample, such as a pretreated GAS sample.

Spectroscopy Measurements

In a preferred embodiment of the present disclosure the GAS data is derived from spectroscopy data. The spectroscopy data may have been obtained by spectroscopically analysis of GAS sample(s). The spectroscopy data may reflect the absorption of the GAS sample in the mid-infrared region (3200-900 cm⁻¹).

The GAS data is preferably derived from measurements of a GAS sample. A GAS sample preferably comprises or consists of gastric aspirates. Alternatively or additionally, a GAS sample may comprise or consist of other bodily fluids, such as pharyngeal secretion (e.g. hypopharyngeal secretions or oropharyngeal secretions) and amniotic fluids, or a combination thereof. Preferably, the GAS sample(s) is substantially dry during the analysis/measurement.

Pretreat

In an embodiment of the present disclosure the GAS sample is, preferably non-invasively, pretreated, prior to spectroscopically analysis. Pretreatment of the GAS sample may for example comprise or consist of centrifugation for formation of a precipitate, and discarding the supernatant. Alternatively or additionally pretreatment may comprise storage, preferably cold storage, such as around 4° C.

In a preferred embodiment of the present disclosure the GAS data and/or lung maturity data is derived from measurements of a bodily fluid, such as gastric aspirates (GAS), pharyngeal secretion (e.g. hypopharyngeal secretions or oropharyngeal secretions), amniotic fluids or GAS, that has been pretreated.

Pretreatment of a bodily fluid may for example comprise or consist of cell lysis, e.g. by mixing with a hypotonic solution, centrifugation for formation of a precipitate, and preferably subsequently discarding the supernatant. Alternatively, or additionally, pretreatment may comprise storage, preferably cold storage, such as around 4° C., or even below the melting point.

Erythrocytes and other cells are often present in GAS. To reduce the contamination of GAS from these sources in order to improve the phospholipid measurements, it has earlier been common practice to centrifuge amniotic fluid or GAS and subsequently discard the precipitate prior to measurement of L/S. However, this procedure reduces the amount of surfactant, resulting in less accurate measurements of lung maturity

Instead, it is a preference that lung maturity data is derived from measurements, such as measurement data, of a bodily fluid, such as GAS, wherein the cells of the bodily fluid has been lysed, such as by mixing with a hypotonic solution. It is further a preference that the bodily fluid subsequently to lysis has been centrifuged at a rotational centrifugal force (RCF) and time selected such that the LBs of the bodily fluid forms a precipitate while the cell fragments, of e.g. lysed cells, and other smaller components, such as salts, remain in the supernatant. An adequate RCF and time may for example be around 4000 g and four minutes. Preferably, the supernatant is discarded following centrifugation. It is further a preference that the measurements of the, preferably diluted and centrifuged, bodily fluid comprise FTIR measurements. The FTIR measurements may thereby be measurements, e.g. dry transmission FTIR, of the LB precipitate for assessment of the lung maturity.

Sphingomyelin is typically sparsely present in the outer membranes of erythrocytes. Therefore, effective removal of erythrocytes before measurements, such as by spectroscopy, e.g. FTIR, may result in slightly increased L/S values, as compared to without removal of erythrocytes. The corresponding L/S cut-off value may as a consequence be higher than as compared to without removal of erythrocytes.

In a preferred embodiment of the present disclosure pretreatment of the bodily fluid comprises dilution with a hypotonic liquid, such as a water solution, e.g. freshwater. Dilution by a low osmolality liquid, such as freshwater, exposes the bodily fluid to hypotonic conditions, causing any present cells, such as erythrocytes, to burst. Preferably the pretreatment further comprises centrifugation, of the diluted bodily fluid. The centrifugation is preferably carried out at a relative centrifugal force, and time, such that the lysates (e.g. ruptured membranes of erythrocytes) and other small components of the solution (e.g. proteins and/or salts) end up in the supernatant while the LBs forms a precipitate, such as around 4000 g for four minutes. Thereby the supernatant It is further a preference that the measurements of the, preferably diluted and centrifuged, bodily fluid comprise FTIR measurements. The FTIR measurements may thereby be a measurement of the LB precipitate for assessment of the lung maturity.

Obtaining GAS Sample

In a preferred embodiment of the present disclosure, the GAS sample has been obtained non-invasively. In a further embodiment of the present disclosure the GAS sample has been collected, from the infant, by a feeding tube in combination with means of displacing GAS through said feeding tube, such as a syringe, or a suction catheter. GAS may for example be collected using a feeding tube attached to a syringe or a suction catheter connected to a tracheal suction set. The feeding tube or suction catheters may be placed as routinely done while establishing nCPAP for respiratory stabilisation or intubation for resuscitation.

Clinical Data

In an embodiment of the present disclosure the clinical data comprises or consists of data selected from the list including birth weight, gestational age, sex, an indicator of whether the infant has been diagnosed with RDS or not, and the severity of RDS (in relevant cases), or a combination thereof. Extreme prematurity and extremely-low-birth-weight have been well established as risk factors for BPD. Gestational age and birth weight are inversely proportional to the incidence of BPD, as well as the severity of the disease. Male infants are known to have a higher risk of developing BPD as compared to females. Additional clinical markers for BPD are known, for example as those outlined in Trembath et al. “Predictors of Bronchopulmonary Dysplasia”, Clin. Perinatol. 2013.

Lung Maturity Data

In a preferred embodiment of the present disclosure the lung maturity data is a binary value (+/−) representing whether the infant has been given, or is to be given, surfactant treatment or not.

If an infant is to be given surfactant treatment, the treatment is ideally started as soon as possible by the administration of a first dose. Preferably the dose should be given within 1 hr of birth but definitely before 2 hours of age. A repeat dose should be given within 4-12 hours if the infant is still intubated and requiring more than 30 to 40% oxygen. Subsequent doses are generally withheld if the infant requires less than 30% oxygen. Typical surfactants include Survanta, Infasurf and Curosurf, associated with specific dosing guidelines.

In an alternative embodiment of the present disclosure lung maturity data is data derived from measurements of a body fluid, for example gastric aspirates (GAS), pharyngeal secretion (e.g. hypopharyngeal secretions or oropharyngeal secretions) and amniotic fluids, or a combination thereof. The lung maturity data may be derived from a lung maturity test, for example the microbubble stability test, the lamellar body counts and/or spectroscopy measurements. Preferably, in the presently disclosed embodiment, the lung maturity data is, or is derived from, spectroscopic data. Thereby, said measurements of the body fluid may be spectroscopic measurements, preferably non-invasive.

Pulmonary surfactant is a surface-active lipoprotein complex produced in type II pneumocytes in the alveoli and secreted as lamellar bodies (LBs) with lung fluid into the amniotic fluid and GAS. The main lipid content of pulmonary surfactant is DPPC. Consequently, the lung maturity data may reflect the content, or the ratio, of a surface-active lung phospholipid, such as lecithin, e.g. dipalmitoylphosphatidylcholine (DPPC), and/or sphingomyelin. The lung maturity data may for example reflect the lecithin/sphingomyelin ratio (L/S).

In an embodiment of the present disclosure the lung maturity data, is derived from, such as comprises or consists of, spectroscopy data, such as mid-infrared spectroscopy data, for assessment of lung maturity. The spectroscopy data may for example have been recorded in the mid-infrared region (3400-900 cm⁻¹). For example by a FTIR spectrometer.

In an embodiment of the present disclosure the lung maturity data comprises one or more measurement values related to the foetal lung maturity of the infant with respect to a cut-off value. For example a measurement value related to the foetal lung maturity, of the infant, that is below (or above) said cut-off value would be associated with a higher risk of diseases related to foetal lung immaturity (such as RDS) while a measurement value above (below) said cut-off value would be associated with a lower risk of diseases related to foetal lung immaturity. The lung maturity data may thereby comprise the difference between the measurement values and the cut-off value or information whether the measurement value is above, or below, said cut-off value. Said cut-off value may be around 3, preferably around 3.05, such as 3.05 in appropriate units (e.g. moles/mol). Said cut-off value may be an L/S value.

The lecithin-sphingomyelin ratio (L/S or L/S ratio) is a test of foetal amniotic fluid to assess foetal lung immaturity. Lungs require surfactants to lower the surface pressure of the alveoli in the lungs. This is especially important for premature babies trying to expand their lungs after birth.

The L/S is a marker of foetal lung maturity. The outward flow of pulmonary secretions from the foetal lungs into the amniotic fluid maintains the level of lecithin and sphingomyelin equally until around 32-33 weeks of gestational age, when the lecithin concentration begins to increase significantly while sphingomyelin remains nearly the same. As such, if a sample of amniotic fluid has a higher ratio, it is indicative of more surfactants in the lungs and that the infant will have less difficulty breathing at birth.

Mathematical Operations

In an embodiment of the present disclosure the GAS data is derived by application of an artificial intelligence (AI) model to the spectroscopy data. The AI model may have been developed by use of training data/outcome data, wherein no a priori knowledge of the relevant molecules and biomarkers are required.

In an embodiment of the present disclosure the GAS data is derived by application of a mathematical operation to the spectroscopy data.

The GAS data may thereby be mathematically derived from spectroscopy data. The mathematical operation may comprise denoising, smoothing, background and baseline corrections, normalization (transforming to a scale of relative intensity), alignment, correction for scatter, such as scattering in NIR, and/or filtering or a combination thereof. The GAS data may thereby be preprocessed in any way.

In general, signal preprocessing is applied to correct and/or remove the contribution of undesired phenomena ranging from stochastic measurement noise to various sources of systematic errors: non-linear instrument responses, shift problems and interfering effects of undesired chemical and physical variations. These operations are also known as denoising, smoothing, background and baseline corrections, normalization (transforming to a scale of relative intensity), alignment (removing horizontal shift), and correction for scatter in near infrared. Moreover, transforming the signal, for example, by derivative operations, can implicitly accomplish normalization, baseline removal and partial band deconvolution. As far as removing horizontal shift is concerned, several algorithms which can aid to remove misalignments have been proposed.

Various filtering methods are known, acting to transform the measured data mathematically into a better version of the same data, leaving out some undesired types of variation, and model-based methods, where the better version is obtained based on a more explicit mathematical model in such a way that the information filtered out is not lost, as statistical estimates of the mathematical parameters involved in the filtering are also obtained.

Among the most used filtering methods for denoising/smoothing, that is, removing uninformative high frequency variation, there are moving average and polynomial Savitsky-Golay filtering, which works on the assumptions that the signal is smooth compared to noise (sum of monotonic functions); noise is mainly uncorrelated and will be eliminated by mild methods. Alternatively high frequency contributions may be removed in frequency (Fourier transform) or wavelet (wavelet transform) domain.

Therefore in an embodiment of the present disclosure the mathematical operation comprises or consists of a 1^{st} order derivative. Alternatively or additionally, the mathematical operation may comprise or consist of a baseline correction algorithm, such as the Savitzky-Golay algorithm.

In an embodiment of the present disclosure the mathematical operation comprises or consists of selecting measurement data at predetermined wavenumbers of the measurement spectrum. Preferably, the predetermined wavenumbers of the measurement spectrum are important for predicting if the infant will develop BPD. Thereby, the measurement data at the predetermined wavenumbers may be indicative of whether the infant will, such as is at risk, of developing BPD. Preferably, the predetermined wavenumbers are selected such that the measurement data corresponding to the predetermined wavenumbers show a difference, preferably a statistically significant difference, difference between infants that develop BPD and infants that do not develop BPD. For example a statistical test may be applied to data acquired, early at birth, of infants, where it is known whether said infants developed BPD or not, to acquire the wavenumbers, the predetermined wavenumbers, that are statistically relevant for predicting BPD. This could thereby be considered to be a training set where the outcome is known, and the relevant wavenumbers for predicting BPD can thereby be acquired. Preferably such a training set is sufficiently large for ensuring that the difference is statistically significant. Such a statistical test may for example be a paired Cox-Wilcoxon test, such as with a two-tailed p-value <0.05.

In an embodiment of the present disclosure the mathematical operation comprises or consists of a partial least square analysis or other methods for multivariate data analysis. PLS may further be used in combination with other classification techniques such as linear discriminant analysis.

In an embodiment of the present disclosure the GAS data is obtained by a process comprising, (non-invasively) obtaining the GAS sample; (optionally) storing the GAS sample; (optionally) pretreating the GAS sample; and obtaining spectroscopy data by analysing/measuring the GAS sample, by spectrometry, such as mid-infrared spectrometry. (optionally) applying one or more mathematical operations to the spectroscopy data. Thereby GAS data is derived from spectroscopy measurements of a GAS sample.

Disease

In an embodiment of the present disclosure BPD is defined as a requirement of supplemental oxygen support at a specific number of days after birth, such as at postnatal day 28. Alternatively, BPD can be defined according to the National Institute of Child Health and Human Development (NICHD) definition from June 2000, comprising a severity-based definition that classifies BPD as mild, moderate or severe based on either postnatal age or PMA. Mild BPD is thereby defined as a need for supplemental oxygen (O₂) throughout the first 28 days but not at 36 weeks PMA or at discharge; moderate BPD as a requirement for 02 throughout the first 28 days plus treatment with <30% O₂ at 36 weeks PMA; severe BPD as a requirement for O₂ throughout the first 28 days plus 30% O₂ and/or positive pressure at 36 weeks PMA. Other definitions, including physiological definitions, exist.

Regardless of which definition of BPD one uses, a period of time is required before the classification of BPD is made. This makes identifying therapies for premature infants at risk of BPD challenging. An infant born at 23-weeks gestation who needs mechanical ventilation at 34 weeks postmenstrual age is likely to develop BPD, as defined as oxygen therapy at 36 weeks. That infant may benefit from strategies that improve short-term outcomes, but which do not reduce the incidence of BPD.

ML Model

In a preferred embodiment of the present disclosure, the analysed data result is obtained by analysing the dataset by a trained machine learning model. Preferably, the trained machine learning model is a supervised trained model, alternatively it may be a supervised and unsupervised trained model.

In an embodiment of the present disclosure the trained model is selected from the list including a support vector machine (SVM), a regression model, an artificial neural network, a decision tree, a genetic algorithm, a Bayesian network, or a combination thereof.

In an embodiment of the present disclosure the prediction comprises or consists of a percentage risk of the infant developing BPD, such as development of BPD according to any definition of BPD. Alternatively, the prediction may further comprise predicting the severity of BPD, for example mild BPD, moderate BPD or severe BPD. The model may thereby predict the development of BPD in an infant, and additionally or alternatively predict the severity of BPD. Predicting the severity of BPD may comprise predicting the severity of BPD in the infant, according to the NICHD definition of BPD, or any other severity-based classification system of BPD.

In an embodiment of the present disclosure the sensitivity of the prediction is at least 70%, more preferably at least 80%, yet even more preferably at least 90%, most preferably at least 95%.

In an embodiment of the present disclosure the specificity of the prediction is at least 70%, more preferably at least 80%, yet even more preferably at least 90%, most preferably at least 95%.

In an embodiment of the present disclosure the specificity and the sensitivity of the prediction is at least 70%, more preferably at least 80%, yet even more preferably at least 90%, most preferably at least 95%.

In a further aspect, the present disclosure relates to the use of a machine learning model for predicting development of BPD in an infant, as disclosed elsewhere herein.

In yet a further aspect, the present disclosure relates to a system for predicting if an infant, early after birth, will develop BPD, the system comprising
a) a memory, and
b) a processing unit that is configured to carry out the method of predicting development of BPD in an infant, as disclosed elsewhere herein, and/or wherein the processing unit is configured to carry out training of a machine learning model for predicting development of BPD in an infant, as disclosed elsewhere herein.

In an embodiment of the present disclosure, the system comprising at least one spectrometry unit for obtaining spectrometry data, such as a spectrometer. Preferably the system is configured to obtain GAS data. The system is preferably comprising a FTIR spectrometer.

In an embodiment of the present disclosure, the system is portable and/or a bedside system. An advantage with the presently disclosed system is that it enables obtaining prediction of BPD early after birth, as the system may be present in the delivery room, or closeby.

Training

The present disclosure further relates to a method for supervised training of a machine learning model for predicting, early after birth, if a subject (e.g. an infant) suffers from, or will develop, bronchopulmonary dysplasia (BPD), the method comprising: obtaining a dataset, comprising information of a number of infants shortly after birth, comprising clinical data; lung maturity data; and gastric aspirate (GAS) data; obtaining outcome data comprising or consisting of information related to if the infants had, or developed, BPD; training a machine learning model, by supervised training, based on the dataset and the outcome data of the infants, to predict, early after birth, if a subject suffers from and/or will develop BPD.

In an embodiment of the present disclosure the subject and/or the infants are preterm born infants, such as born before 37 weeks of pregnancy are completed. In a preferred embodiment of the present disclosure, the infant is a preterm born infant, such as an infant born before 37 weeks of pregnancy are completed. Preterm birth, also known as premature birth, is the birth of a baby at fewer than 37 weeks' gestational age, as opposed to the usual about 40 weeks. The infant may however be born at an earlier stage of pregnancy, such as less than 35 weeks' gestational age, or even less than 30 weeks' gestational age. The risk of development of BPD is higher at a lower gestational age.

It is a preference that the dataset comprises or consists of data obtained within 24 hours after birth, such as at birth. The earlier prediction of development of BPD in an infant is made, the earlier a targeted intervention can be started, having the potential to significantly improve outcomes. The early intervention may comprise preventative and targeted prophylactic, therapeutic intervention with surfactant and new medicaments, and/or the mode of ventilation. Various strategies for treatment and preventive therapy of BPD are known to a person skilled in the art.

GAS Data

In an embodiment of the present disclosure the GAS data, is derived from, such as comprises or consists of, spectroscopy data, such as mid-infrared spectroscopy data. The GAS data may for example be derived from, or comprise, spectroscopy data in the spectrum between 900-3400 cm⁻¹, such as between 900-1800 cm⁻¹ and between 2800-3400 cm⁻¹. Spectroscopy measurements of GAS, for example by FTIR spectroscopy, typically enable derivation of a highly detailed digital fingerprint of the foetal lung biochemistry. Thereby, GAS data may comprise FTIR spectral wavelengths and/or absorption intensities and may, combined with other markers, be evaluated for the prediction of BPD.

In an embodiment of the present disclosure, an AI model is trained, based on outcome data, to select data points or spectral lines of a gastric aspirate measurement, wherein the data points or spectral lines are selected to most accurately distinguish between infants that develop BPD and those who do not develop BPD. As such, the training of the machine learning model may not require a priori knowledge of the relevant molecules and biomarkers of the gastric aspirate.

In an embodiment of the present disclosure the GAS data is derived from, such as comprises or consists of, one or more absorption and/or one or more transmission spectra. The GAS data may consist of data derived from a single spectroscopy measurement, or the GAS data may comprise data derived from multiple spectroscopy measurements. Furthermore, the multiple measurements may have been carried out on different types of bodily fluids. In a preferred embodiment of the present disclosure the GAS data is derived from measurements of a GAS sample, such as a pretreated GAS sample.

Spectroscopy Measurements

In a preferred embodiment of the present disclosure the GAS data is derived from spectroscopy data. The spectroscopy data may have been obtained by spectroscopically analysis of GAS sample(s). The spectroscopy data may reflect the absorption of the GAS sample in the mid-infrared region (3200-900 cm⁻¹).

The GAS data is preferably derived from measurements of a GAS sample. The GAS sample preferably comprise or consists of gastric aspirates. Alternatively or additionally, a GAS sample may comprise or consist of other bodily fluids, such as pharyngeal secretion (e.g. hypopharyngeal secretions or oropharyngeal secretions) and amniotic fluids, or a combination thereof. Preferably, the GAS sample(s) is substantially dry during the analysis/measurement.

Pretreat

In an embodiment of the present disclosure the GAS sample is, preferably non-invasively, pretreated, prior to spectroscopically analysis. Pretreatment of the GAS sample may for example comprise or consist of centrifugation for formation of a precipitate, and discarding the supernatant. Alternatively or additionally pretreatment may comprise storage, preferably cold storage, such as around 4° C.

In a preferred embodiment of the present disclosure the GAS data and/or lung maturity data is derived from measurements of a bodily fluid, such as gastric aspirates (GAS), pharyngeal secretion (e.g. hypopharyngeal secretions or oropharyngeal secretions), amniotic fluids or GAS, that has been pretreated.

Pretreatment of a bodily fluid may for example comprise or consist of cell lysis, e.g. by mixing with a hypotonic solution, centrifugation for formation of a precipitate, and preferably subsequently discarding the supernatant. Alternatively, or additionally, pretreatment may comprise storage, preferably cold storage, such as around 4° C., or even below the melting point.

Erythrocytes and other cells are often present in GAS. To reduce the contamination of GAS from these sources in order to improve the phospholipid measurements, it has earlier been common practice to centrifuge amniotic fluid or GAS and subsequently discard the precipitate prior to measurement of L/S. However, this procedure reduces the amount of surfactant, resulting in less accurate measurements of lung maturity

Instead, it is a preference that lung maturity data is derived from measurements, such as measurement data, of a bodily fluid, such as GAS, wherein the cells of the bodily fluid has been lysed, such as by mixing with a hypotonic solution. It is further a preference that the bodily fluid subsequently to lysis has been centrifuged at a rotational centrifugal force (RCF) and time selected such that the LBs of the bodily fluid forms a precipitate while the cell fragments, of e.g. lysed cells, and other smaller components, such as salts, remain in the supernatant. An adequate RCF and time may for example be around 4000 g and four minutes. Preferably, the supernatant is discarded following centrifugation. It is further a preference that the measurements of the, preferably diluted and centrifuged, bodily fluid comprise FTIR measurements. The FTIR measurements may thereby be measurements, e.g. dry transmission FTIR, of the LB precipitate for assessment of the lung maturity.

Sphingomyelin is typically sparsely present in the outer membranes of erythrocytes. Therefore, effective removal of erythrocytes before measurements, such as by spectroscopy, e.g. FTIR, may result in slightly increased L/S values, as compared to without removal of erythrocytes. The corresponding L/S cut-off value may as a consequence be higher than as compared to without removal of erythrocytes.

In a preferred embodiment of the present disclosure pretreatment of the bodily fluid comprises dilution with a hypotonic liquid, such as a water solution, e.g. freshwater. Dilution by a low osmolality liquid, such as freshwater, exposes the bodily fluid to hypotonic conditions, causing any present cells, such as erythrocytes, to burst. Preferably the pretreatment further comprises centrifugation, of the diluted bodily fluid. The centrifugation is preferably carried out at a relative centrifugal force, and time, such that the lysates (e.g. ruptured membranes of erythrocytes) and other small components of the solution (e.g. proteins and/or salts) end up in the supernatant while the LBs forms a precipitate, such as around 4000 g for four minutes. Thereby the supernatant It is further a preference that the measurements of the, preferably diluted and centrifuged, bodily fluid comprise FTIR measurements. The FTIR measurements may thereby be a measurement of the LB precipitate for assessment of the lung maturity.

Obtaining GAS Sample

In a preferred embodiment of the present disclosure, the GAS sample has been obtained non-invasively. In a further embodiment of the present disclosure the GAS sample has been collected, from the infant, by a feeding tube in combination with means of displacing GAS through said feeding tube, such as a syringe, or a suction catheter. GAS may for example be collected using a feeding tube attached to a syringe or a suction catheter connected to a tracheal suction set. The feeding tube or suction catheters may be placed as routinely done while establishing nCPAP for respiratory stabilisation or intubation for resuscitation.

Clinical Data

In an embodiment of the present disclosure the clinical data comprises or consists of data selected from the list including birth weight, gestational age, sex, an indicator of whether the infant has been diagnosed with RDS or not, and the severity of RDS (in relevant cases), or a combination thereof. Extreme prematurity and extremely-low-birth-weight have been identified as risk factors for BPD. Gestational age and birth weight are inversely proportional to the incidence of BPD, as well as the severity of the disease. Male infants are known to have a higher risk of developing BPD as compared to females. Additional clinical markers for BPD are known, for example as those outlined in Trembath et al. “Predictors of Bronchopulmonary Dysplasia”, Clin. Perinatol. 2013.

Lung Maturity Data

In a preferred embodiment of the present disclosure the lung maturity data is a binary value (+/−) representing whether the infant has been given, or is to be given, surfactant treatment or not.

If an infant is to be given surfactant treatment, the treatment is ideally started as soon as possible by the administration of a first dose. Preferably the dose should be given within 1 hr of birth but definitely before 2 hours of age. A repeat dose should be given within 4-12 hours if the infant is still intubated and requiring more than 30 to 40% oxygen. Subsequent doses are generally withheld if the infant requires less than 30% oxygen. Typical surfactants include Survanta, Infasurf and Curosurf, associated with specific dosing guidelines.

In an alternative embodiment of the present disclosure lung maturity data is data derived from measurements of a body fluid, for example gastric aspirates (GAS), pharyngeal secretion (e.g. hypopharyngeal secretions or oropharyngeal secretions) and amniotic fluids, or a combination thereof. The lung maturity data may be derived from a lung maturity test, for example the microbubble stability test, the lamellar body counts and/or spectroscopy measurements. Preferably, in the presently disclosed embodiment, the lung maturity data is, or is derived from, spectroscopic data. Thereby, said measurements of the body fluid may be spectroscopic measurements, preferably non-invasive.

Pulmonary surfactant is a surface-active lipoprotein complex produced in type II pneumocytes in the alveoli and secreted as lamellar bodies (LBs) with lung fluid into the amniotic fluid and GAS. The main lipid content of pulmonary surfactant is DPPC. Consequently, the lung maturity data may reflect the content, or the ratio, of a surface-active lung phospholipid, such as lecithin, e.g. dipalmitoylphosphatidylcholine (DPPC), and/or sphingomyelin. The lung maturity data may for example reflect the lecithin/sphingomyelin ratio (L/S).

In an embodiment of the present disclosure the lung maturity data, is derived from, such as comprises or consists of, spectroscopy data, such as mid-infrared spectroscopy data, for assessment of lung maturity. The spectroscopy data may for example have been recorded in the mid-infrared region (3400-900 cm⁻¹). For example by a FTIR spectrometer.

In an embodiment of the present disclosure the lung maturity data comprises one or more measurement values related to the foetal lung maturity of the infant with respect to a cut-off value. For example a measurement value related to the foetal lung maturity, of the infant, that is below (or above) said cut-off value would be associated with a higher risk of diseases related to foetal lung immaturity (such as RDS) while a measurement value above (below) said cut-off value would be associated with a lower risk of diseases related to foetal lung immaturity. The lung maturity data may thereby comprise the difference between the measurement values and the cut-off value or information whether the measurement value is above, or below, said cut-off value. Said cut-off value may be around 3, preferably around 3.05, such as 3.05 in appropriate units (e.g. moles/mol). Said cut-off value may be an L/S value.

The lecithin-sphingomyelin ratio (L/S or L/S ratio) is a test of foetal amniotic fluid to assess foetal lung immaturity. Lungs require surfactants to lower the surface pressure of the alveoli in the lungs. This is especially important for premature babies trying to expand their lungs after birth.

The L/S is a marker of foetal lung maturity. The outward flow of pulmonary secretions from the foetal lungs into the amniotic fluid maintains the level of lecithin and sphingomyelin equally until around 32-33 weeks of gestational age, when the lecithin concentration begins to increase significantly while sphingomyelin remains nearly the same. As such, if a sample of amniotic fluid has a higher ratio, it is indicative of more surfactants in the lungs and that the infant will have less difficulty breathing at birth.

Mathematical Operations

In an embodiment of the present disclosure, an AI model is trained, based on outcome data, to select data points or spectral lines of a gastric aspirate measurement, wherein the data points or spectral lines are selected to most accurately distinguish between infants that develop BPD and those who do not develop BPD. As such, the training of the machine learning model may not require a priori knowledge of the relevant molecules and biomarkers of the gastric aspirate.

In an embodiment of the present disclosure the GAS data is derived by application of a mathematical operation to the spectroscopy data. The GAS data may thereby be mathematically derived from spectroscopy data. The mathematical operation may comprise denoising, smoothing, background and baseline corrections, normalization (transforming to a scale of relative intensity), alignment, correction for scatter, such as scattering in NIR, and/or filtering or a combination thereof. The GAS data may thereby be preprocessed in any way.

In general, signal preprocessing is applied to correct and/or remove the contribution of undesired phenomena ranging from stochastic measurement noise to various sources of systematic errors: non-linear instrument responses, shift problems and interfering effects of undesired chemical and physical variations. These operations are also known as denoising, smoothing, background and baseline corrections, normalization (transforming to a scale of relative intensity), alignment (removing horizontal shift), and correction for scatter in near infrared. Moreover, transforming the signal, for example, by derivative operations, can implicitly accomplish normalization, baseline removal and partial band deconvolution. As far as removing horizontal shift is concerned, several algorithms which can aid to remove misalignments have been proposed.

Various filtering methods are known, acting to transform the measured data mathematically into a better version of the same data, leaving out some undesired types of variation, and model-based methods, where the better version is obtained based on a more explicit mathematical model in such a way that the information filtered out is not lost, as statistical estimates of the mathematical parameters involved in the filtering are also obtained.

Among the most used filtering methods for denoising/smoothing, that is, removing uninformative high frequency variation, there are moving average and polynomial Savitsky-Golay filtering, which works on the assumptions that the signal is smooth compared to noise (sum of monotonic functions); noise is mainly uncorrelated and will be eliminated by mild methods. Alternatively high frequency contributions may be removed in frequency (Fourier transform) or wavelet (wavelet transform) domain.

Therefore in an embodiment of the present disclosure the mathematical operation comprises or consists of a 1^{st} order derivative. Alternatively or additionally, the mathematical operation may comprise or consist of a baseline correction algorithm, such as the Savitzky-Golay algorithm.

In an embodiment of the present disclosure the mathematical operation comprises or consists of selecting measurement data at predetermined wavenumbers of the measurement spectrum. Preferably, the predetermined wavenumbers of the measurement spectrum are important for predicting if the infant will develop BPD. Thereby, the measurement data at the predetermined wavenumbers may be indicative of whether the infant will, such as is at risk, of developing BPD. Preferably, the predetermined wavenumbers are selected such that the measurement data corresponding to the predetermined wavenumbers show a statistical significance or a difference, preferably a statistical significance difference, between infants that develop BPD and infants that do not develop BPD. For example a statistical test may be applied to data acquired, early at birth, of infants, where it is known whether said infants developed BPD or not, to acquire the wavenumbers, the predetermined wavenumbers, that are statistically relevant for predicting BPD. This could thereby be considered to be a training set where the outcome is known, and the relevant wavenumbers for predicting BPD can thereby be acquired. Preferably such a training set is sufficiently large for ensuring that the difference is statistically significant. Such a statistical test may for example be a paired Cox-Wilcoxon test, such as with a two-tailed p-value <0.05.

In an embodiment of the present disclosure the mathematical operation comprises or consists of a partial least square analysis or other methods for multivariate data analysis. PLS may further be used in combination with other classification techniques such as linear discriminant analysis.

In an embodiment of the present disclosure the GAS data is obtained by a process comprising, (non-invasively) obtaining the GAS sample; (optionally) storing the GAS sample; (optionally) pretreating the GAS sample; and obtaining spectroscopy data by analysing/measuring the GAS sample, by spectrometry, such as mid-infrared spectrometry. (optionally) applying one or more mathematical operations to the spectroscopy data. Thereby GAS data is derived from spectroscopy measurements of a GAS sample.

Disease

In an embodiment of the present disclosure the classification of BPD is defined as a subject requiring supplemental oxygen support at a specific number of days after birth, typically at postnatal day 28. Alternatively, BPD can be defined according to the National Institute of Child Health and Human Development (NICHD) definition from June 2000, comprising a severity-based definition that classifies BPD as mild, moderate or severe based on either postnatal age or PMA. Mild BPD is thereby defined as a need for supplemental oxygen (O₂) throughout the first 28 days but not at 36 weeks PMA or at discharge; moderate BPD as a requirement for 02 throughout the first 28 days plus treatment with <30% O₂ at 36 weeks PMA; severe BPD as a requirement for O₂ throughout the first 28 days plus 30% O₂ and/or positive pressure at 36 weeks PMA. Other definitions, including physiological definitions, exist.

Regardless of which definition of BPD one uses, a period of time is required before the classification of BPD is made. This makes identifying therapies for premature infants at risk of BPD challenging. An infant born at 23-weeks gestation who needs mechanical ventilation at 34 weeks postmenstrual age is likely to develop BPD, as defined as oxygen therapy at 36 weeks. That infant may benefit from strategies that improve short-term outcomes, but which do not reduce the incidence of BPD.

ML Model

In a preferred embodiment of the present disclosure, the analysed data result is obtained by analysing the dataset by a trained machine learning model. Preferably, the trained machine learning model is a supervised trained model, alternatively it may be a supervised and unsupervised trained model.

In an embodiment of the present disclosure the trained model is selected from the list including a support vector machine (SVM), a regression model, an artificial neural network, a decision tree, a genetic algorithm, a Bayesian network, or a combination thereof.

In an embodiment of the present disclosure the prediction comprises or consists of a percentage risk of the infant developing BPD, such as development of BPD according to any definition of BPD. Alternatively, the prediction may further comprise predicting the severity of BPD, for example mild BPD, moderate BPD or severe BPD. The model may thereby predict the development of BPD in an infant, and additionally or alternatively predict the severity of BPD. Predicting the severity of BPD may comprise predicting the severity of BPD in the infant, according to the NICHD definition of BPD, or any other severity-based classification system of BPD.

In an embodiment of the present disclosure the sensitivity of the prediction is at least 70%, more preferably at least 80%, yet even more preferably at least 90%, most preferably at least 95%.

In an embodiment of the present disclosure the specificity of the prediction is at least 70%, more preferably at least 80%, yet even more preferably at least 90%, most preferably at least 95%.

In an embodiment of the present disclosure the specificity and the sensitivity of the prediction is at least 70%, more preferably at least 80%, yet even more preferably at least 90%, most preferably at least 95%.

In an embodiment of the present disclosure the trained machine learning model is evaluated. The evaluation of the trained machine learning model may be carried out by a dataset and an outcome data distinct from those used during the training of the machine learning model.

The present disclosure further relates to a system for predicting if an infant, early after birth, will develop BPD, the system comprising a memory, and a processing unit that is configured to carry out the method for predicting a risk of an infant developing bronchopulmonary dysplasia (BPD), as described elsewhere herein and/or the method for supervised training of a machine learning model for predicting, early after birth, if an infant suffers from, or will develop, bronchopulmonary dysplasia (BPD) as disclosed elsewhere herein.

In an embodiment of the present disclosure the system further comprising at least one spectrometry unit for obtaining spectrometry data, such as a spectrometer. Preferably said spectrometer is configured to obtain spectrometry data from a GAS sample and to provide said spectrometry data to the processing unit for processing of said spectrometry data. The system may thereby comprise means for providing said spectrometry data to the processing unit and/or the memory. Preferably said system further comprises a power source.

### EXAMPLES

### Example 1—Training of a Machine Learning Algorithm for Predicting Development of BPD in an Infant

BPD Definition

The Consensus BPD definition from the US National Institutes of Health (NIH) was applied. For infants born at gestational age (GA)<32 weeks, BPD referred to the requirement of oxygen support for at least 28 days (all severities of BPD) supplemented with an assessment at 36 weeks (moderate to severe BPD) and at 40 weeks (severe BPD).

Participants

Premature infants born between 24 and 31 completed gestational weeks were eligible to participate. The infants enrolled in the study were treated as described in Heiring et al. “Predicting respiratory distress syndrome at birth using a fast test based on spectroscopy of gastric aspirates: 2. Clinical part.” Acta Paediatr. 2019, with antenatal steroids and very early nasal-CPAP when possible. Surfactant (Curosurf R) was administered following the European Consensus Guidelines on the Management of RDS as INSURE (Intubation-Surfactant-Extubation) or nasal-CPAP and surfactant administered by a thin catheter.

Sampling of GAS and Spectroscopy

Sampling of GAS at birth (0.3-2.5 mL) was collected using a feeding tube attached to a syringe or a suction catheter connected to a tracheal suction set. The feeding tube or suction catheters were placed as routinely done while establishing nCPAP for respiratory stabilisation or intubation for resuscitation.

Gastric aspirates obtained immediately after birth were stored at 4-5° C. and analysed by FTIR spectroscopy within 10 days.

The FTIR spectroscopy was performed by dry transmission, and the spectroscopic signal was enhanced by concentrating the surfactant thus avoiding the interference of proteins, salts or flocculent protein clots (e.g. mucus).

GAS (200 μL) was diluted fourfold with water and centrifuged at 4000 g for four minutes. After removal of the supernatant, the samples were suspended in 100 μL of water and split into 50 μL aliquots. 50 μL of sample was measured by FTIR analyses performed by dry transmission on CaF₂ windows (1 mm thick and 13 mm diameter, Chrystran.com). The samples (50 μL) were applied onto the CaF₂ and dried on a hotplate (90° C.). The FTIR spectra were measured by a Bruker Tensor 27, equipped with a DTGS detector (60 scans and a resolution of 4 cm⁻¹).

Basic Method Development Principles

A data-driven approach was employed to develop a software algorithm capable of predicting BPD. Clinical data and lung maturity data (+/− surfactant treatment) available near the time of birth were combined with FTIR spectral data of GAS resulting in the creation of highly complex multivariate datasets. These datasets were analysed using AI and corrected to the clinical development of BPD.

Statistical Analysis

Clinical data points correlated to BPD were determined by t-test for continuous variables and chi-square test for categorical variables. Paired Cox-Wilcoxon test was used for FTIR spectral data analysis. Two-tailed p-values <0.05 were considered to indicate statistical significance.

FTIR Spectral Data

The FTIR spectral analysis range was 900-3400 cm⁻¹. Baseline was corrected using the Savitzky-Golay algorithm and the 1^{st} derivative was used for spectral data analysis. The Cox-Wilcoxon test was used to further select the most important variables and 43 wavenumbers were selected out of 1.200.

Model Development

Partial Least Square (PLS)

The PLS algorithm used was similar to that used in Hoskuldsson, “Common framework for linear regression”, Chemometrics and Intelligent Laboratory Systems, 2015. The score plots produced by PLS in combination with other classification techniques such as linear discriminant analysis have in many cases been proved to separate samples for better determination.

Software

R studio (Microsoft R open) software was used. A SVM model was built using the Kernlab package written in R programming language. The validation of the model performance in the training sample was 7-fold cross validation repeated 500 times. The criterion for selecting the best parameters was the minimization of classification error. Additionally, the mean sensitivity and specificity of the cross validation was calculated. The sensitivity was defined as the percentage of the correct prediction of the infants with BPD and the specificity as the correct prediction of the infants who did not develop BPD.

Results

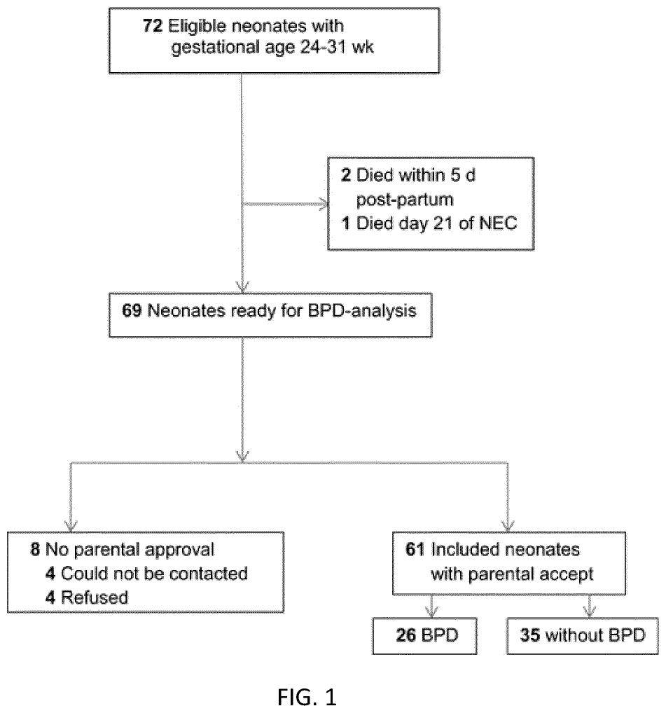

Of the 72 eligible infants 2 died early after birth and in 9 cases parental approvals were not obtained. Thus, 61 very preterm infants were included in the study as shown in FIG. 1. The clinical characteristics of the included infants are presented in Table 1.

TABLE 1
Characteristics of included neonates

| | Cohort (No. = 61) | |
|---|---|---|
| Clinical Variable | | |
| Gestational age, wk^{a} | 28.5 | (24.3-31.7) |
| Birth weight, g^{a} | 1.014 | (525-2.110) |
| Male^{b} | 35 | (57) |
| Antenatal steroid^{b} | 58 | (95) |
| 2 doses^{b} | 48 | (83) |
| Caesarean section^{b} | 43 | (70) |
| Mechanical ventilation | | |
| within 5 days post-partum^{b} | 14 | (23) |
| Apgar 5 min^{a} | 9.2 | (4-10) |
| Respiratory distress syndrome^{b} | 39 | (64) |
| Moderate-severe | 28 | (46) |
| Surfactant treatment^{b} | 27 | (44) |
| Time to surfactant treatment, h^{a} | 5.8 | (0.1-33) |

^{a}Median (range)
^{b}No. (%)

Twenty-six (43%) developed BPD and 35 (57%) did not develop BPD. Ten of the infants with BPD also had a need for supplemental oxygen at week 36 and 2 still needed supplemental oxygen week 40.

A majority 39 (64%) of the included 61 infants had either BPD combined with RDS (n=22), or no-BPD and no RDS (n=18). Whereas, 4 BPD infants had no RDS and 17 infants with no BPD had RDS (Table 2).

TABLE 2
BPD versus RDS

| | BPD | no BPD |
|---|---|---|
| | (No. of infants) | (No. of infants) |
| RDS | 22 | 17 |
| No RDS | 4 | 18 |

The 26 infants with BPD had a median birth weight (BW) of 850 g, a median gestational age (GA) of 27.3 weeks and 20 (77%) were treated with surfactant. The 35 infants with no BPD had median BW of 1.356 g, a median GA of 30.1 weeks and 7 (20%) were treated with surfactant. BW and GA were significantly lower for infants with BPD than for infants without BPD, p<0.001 and more infants with BPD than with no BPD were treated with surfactant, p<0.001. Surfactant was given after 5.8 hours in median and latest after 33 hours (Table 1). BW, GA and surfactant treatment are important factors correlated to the development of BPD and by analysing them using a logistic regression model the sensitivity and specificity were 74% and 82% respectively. Similar data were obtained by applying SVM resulting in 76% sensitivity and 82% specificity.

The FTIR spectral data analysis of GAS resulted in the identification of the most important wavenumbers for classification. In order to reveal significant differences in the wavenumbers between BPD and no BPD a paired Cox-Wilcoxon test was applied. In total, 43 wavenumbers were selected from the selected FTIR spectral dataset.

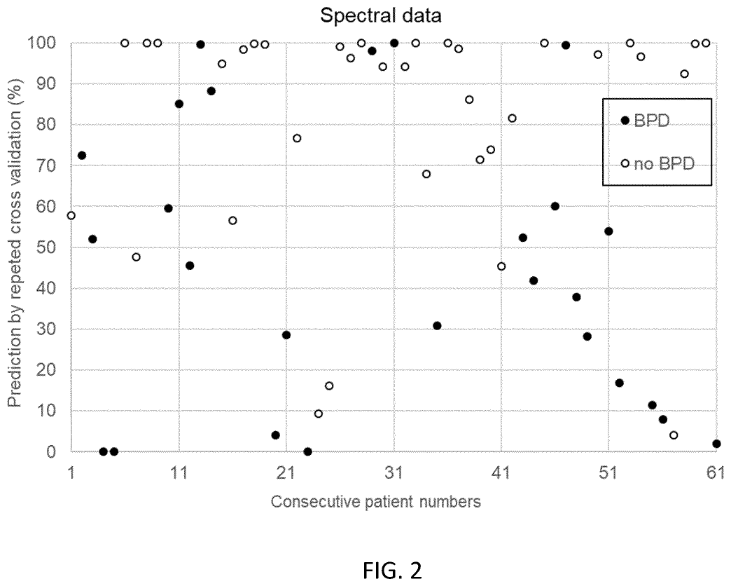

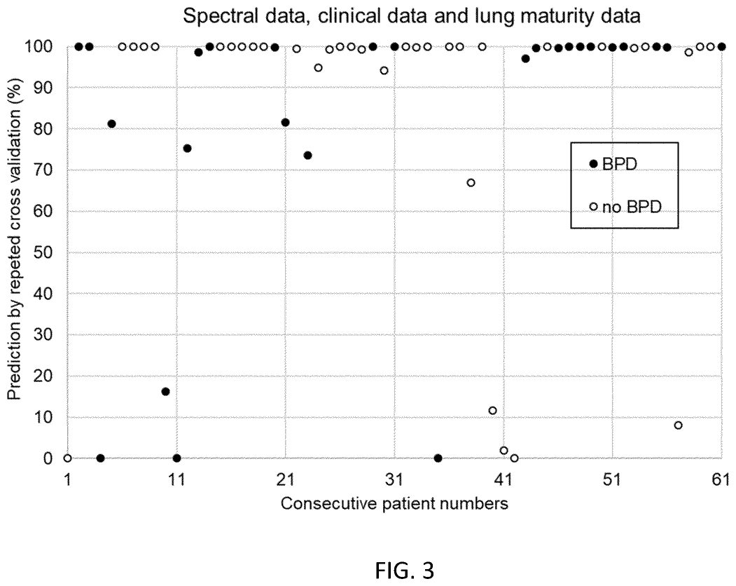

Prediction of BPD from FTIR spectral data of GAS samples alone are shown in FIG. 2 whereas FIG. 3 illustrates how well BPD is predicted when FTIR spectral data, clinical data (in the form of birth weight and gestational age) and lung maturity data (in the form of whether surfactant treatment has been carried out or nor) were combined in the analysis. Predictions were considered accurate in samples where repeated cross validation outcomes exceeded 50%. Five samples (numbers 01, 40, 41, 42, 57) from infants with no BPD treated with surfactant early after birth were difficult to classify from the combined dataset of FTIR spectral data, clinical data and lung maturity data (FIG. 3). PLS analyses showed that the best prediction of these samples was obtained from analysis of FTIR spectral data only. As seen from FIG. 2, sample numbers 01, 40 and 42 were predicted well in more than 50% of the cross validations and prediction of sample 41 was lifted from 2 to 46%. GAS sample numbers 04, 10, 11 and 35 from infants with BPD and no RDS were also difficult to classify. Two of these infants, number 10 and 11, could only be classified by the FTIR spectral data (FIG. 2).

By incorporating FTIR spectral data analyses with the clinical data and lung maturity data (BW, GA and surfactant treatment) into the linear SVM analysis the sensitivity increased from 76% to 86% and the specificity from 82% to 85% following cross validation. Using the parameters selected by cross validation, the fitting model was finally calculated for the 61 samples revealing a sensitivity and specificity of 88% and 91% respectively. One GAS sample was contaminated with pus. However, it was still possible to measure the sample using FTIR and correctly predict BPD.

Conclusions

The study demonstrated that it was possible to predict BPD at birth by applying AI to analyse unique multivariate datasets combining clinical data and FTIR spectral data of GAS. Further development and validation of the predictive BPD algorithm is planned including data aggregation, blind testing and clinical studies.

Items
1. A computer-implemented method for predicting a risk of an infant developing bronchopulmonary dysplasia (BPD), the method comprising the steps of:
   obtaining a dataset, of the infant, comprising
      clinical data;
      lung maturity data; and
      gastric aspirate (GAS) data;
   analysing said dataset, thereby obtaining an analysed data result; and
   based on said analysed data result predicting the risk of the infant developing BPD.
2. The computer-implemented method according to any one of the preceding items, wherein the analysed data result is obtained by analysing the dataset by a trained machine learning model.
3. The computer-implemented method according to any one of the preceding items, wherein the infant is a preterm born infant, such as an infant born before 37 weeks of pregnancy are completed.
4. The computer-implemented method according to any one of the preceding items, wherein the dataset comprises or consists of data obtained within 48 hours after birth, preferably within 36 hours after birth.
5. The computer-implemented method according to any one of the preceding items, wherein the clinical data comprises or consists of data, of the infant, selected from the list including birth weight, gestational age, if the infant has been diagnosed with RDS and/or the severity of RDS.
6. The computer-implemented method according to any one of the preceding items, wherein the lung maturity data is data indicative of the maturity of the lungs of the infant, and/or an indicator representing whether the infant has been given, or is to be given, surfactant treatment or not, or a combination thereof.
7. The computer-implemented method according to any one of the preceding items, wherein the lung maturity data, is derived from, such as comprises or consists of, spectroscopy data, such as mid-infrared spectroscopy data, for assessment of lung maturity.
8. The computer-implemented method according to any one of the preceding items, wherein the lung maturity data is indicative of the lecithin-sphingomyelin ratio.
9. The computer-implemented method according to any one of the preceding items, wherein the lung maturity data is obtained, non-invasively, by spectroscopic analysis of GAS sample(s), such as mid-infrared spectroscopic analysis.
10. The computer-implemented method according to any one of the preceding items, wherein the lung maturity data is derived from measurement data of a bodily fluid sample, comprising GAS, pharyngeal secretion and/or amniotic fluid.
11. The computer-implemented method according to item 10, wherein said bodily fluid sample has been pretreated prior to measurements, said pretreatment comprising
   a) lysing cells present in the bodily fluid sample, such as by mixing with freshwater;
   b) centrifugation of the lysed sample, at a rotational centrifugal force (RCF) and time selected such that LBs of the bodily fluid sample forms a precipitate while cell fragments, of e.g. lysed cells, and other smaller components, such as salts, remain in a supernatant.
   c) (optionally) discarding said supernatant
12. The computer-implemented method according to item 11, wherein the lung maturity data is derived from measurements of the precipitate, such as dry transmission FTIR measurements.
13. The computer-implemented method according to any one of the preceding items, wherein the GAS data, is derived from, such as comprises or consists of, spectroscopy data, such as mid-infrared spectroscopy data.
14. The computer-implemented method according to any one of the preceding items, wherein the GAS data is derived from spectroscopy data in the spectrum between 900-3400 cm⁻¹, such as between 900-1800 cm⁻¹ and between 2800-3400 cm⁻¹.
15. The computer-implemented method according to any one of the preceding items, wherein the GAS data is derived from, such as comprises or consists of, one or more absorption and/or transmission spectra.
16. The computer-implemented method according to any one of the preceding items, wherein the GAS data is derived from spectroscopy data obtained by spectroscopically analysis of GAS sample(s).
17. The computer-implemented method according to any one of the preceding items, wherein the GAS sample(s) is substantially dry during the analysis.
18. The computer-implemented method according to any one of the preceding items, wherein the GAS sample is pretreated, prior to spectroscopically analysis.
19. The computer-implemented method according to any one of the preceding items, wherein the pretreatment comprises or consists of centrifugation for formation of a precipitate, and discarding the supernatant.
20. The computer-implemented method according to any one of the preceding items, wherein the GAS sample has been collected, from the infant, by a feeding tube in combination with means of displacing GAS through said feeding tube, such as a syringe, or a suction catheter.
21. The computer-implemented method according to any one of the preceding items, wherein the GAS data is derived by application of a mathematical operation to the spectroscopy data.
22. The computer-implemented method according to any one of the preceding items, wherein the mathematical operation comprises or consists of a 1^{st} order derivative.
23. The computer-implemented method according to any one of the preceding items, wherein the mathematical operation comprises or consists of a baseline correction algorithm, such as the Savitzky-Golay algorithm.
24. The computer-implemented method according to any one of the preceding items, wherein the mathematical operation comprises or consists of selecting predetermined wavenumbers of the spectrum.
25. The computer-implemented method according to item 24, wherein the predetermined wavenumbers show a statistical significant difference between infants that develop BPD and infants that do not develop BPD.
26. The computer-implemented method according to item 25, wherein the statistical significant difference is based on a statistical test, such as the paired Cox-Wilcoxon test, with a two-tailed p-value <0.05.
27. The computer-implemented method according to any one of the preceding items, wherein the mathematical operation comprises or consists of a partial least square analysis.
28. The computer-implemented method according to any one of the preceding items, wherein the GAS data is obtained by a process comprising:
   a) (optionally) obtaining the GAS sample;
   b) (optionally) storing the GAS sample;
   c) (optionally) pretreating the GAS sample; and
   d) obtaining spectroscopy data by analysing the GAS sample, by spectrometry, such as mid-infrared spectrometry.
   e) (optionally) applying one or more mathematical operations to the spectroscopy data.
29. The computer-implemented method according to any one of the preceding items, wherein BPD is defined as a requirement of supplemental oxygen support at a specific number of days after birth, such as at postnatal day 28.
30. The computer-implemented method according to any one of the preceding items, wherein the trained model is a supervised trained model or a supervised and unsupervised trained model.
31. The computer-implemented method according to any one of the preceding items, wherein the trained model is selected from the list including a support vector machine (SVM), a regression model, an artificial neural network, a decision tree, a genetic algorithm, a Bayesian network, or a combination thereof.
32. The computer-implemented method according to any one of the preceding items, wherein the prediction comprises or consists of a percentage risk of the infant developing BPD.
33. The computer-implemented method according to any one of the preceding items, wherein the sensitivity of the prediction is at least 70%.
34. The computer-implemented method according to any one of the preceding items, wherein the specificity of the prediction is at least 70%.
35. A method for supervised training of a machine learning model for predicting, early after birth, if a subject suffers from, or will develop, bronchopulmonary dysplasia (BPD), the method comprising:
   a) obtaining a dataset, comprising information of a number of infants shortly after birth, comprising
      clinical data;
      lung maturity data; and
      gastric aspirate (GAS) data;
   b) obtaining outcome data comprising or consisting of information related to if the infants had, or developed, BPD;
   c) training a machine learning model, by supervised training, based on the dataset and the outcome data of the infants, to predict, early after birth, if a subject suffers from and/or will develop BPD.
36. The method for supervised training of a machine learning model according to item 35, wherein the subject and/or the infants are preterm born infants, such as born before 37 weeks of pregnancy are completed.
37. The method for supervised training of a machine learning model according to any of items 35-36, wherein the dataset comprises or consists of data obtained within 24 hours after birth, such as at birth.
38. The method for supervised training of a machine learning model according to any of items 35-37, wherein the clinical data comprises or consists of data selected from the list including birth weight, gestational age, if the infant has been diagnosed with RDS, the severity of RDS (in relevant cases), or a combination thereof.
39. The method for supervised training of a machine learning model according to any of items 35-38, wherein the lung maturity data is data indicative of the maturity of the lungs of the infant, and/or a binary value (+/−) representing whether the infant has been given, or is to be given, surfactant treatment or not, or a combination thereof
40. The method for supervised training of a machine learning model according to any of items 35-39, wherein the lung maturity data, is derived from, such as comprises or consists of, spectroscopy data, such as mid-infrared spectroscopy data, for assessment of lung maturity.
41. The method for supervised training of a machine learning model according to any of items 35-40, wherein the lung maturity data is indicative of the lecithin-sphingomyelin ratio.
42. The method for supervised training of a machine learning model according to any of items 35-41, wherein the lung maturity data is obtained, non-invasively, by spectroscopic analysis of GAS sample(s), such as mid-infrared spectroscopic analysis.
43. The method for supervised training of a machine learning model according to any of items 35-42, wherein the lung maturity data is derived from measurement data of a bodily fluid sample, comprising or consisting of GAS, pharyngeal secretion and/or amniotic fluid or a combination thereof.
44. The method for supervised training of a machine learning model according to item 43, wherein said bodily fluid sample has been pretreated prior to measurements, said pretreatment comprising
   a) lysing cells present in the bodily fluid sample, such as by mixing with freshwater;
   b) centrifugation of the lysed sample, at a rotational centrifugal force (RCF) and time selected such that LBs of the bodily fluid sample forms a precipitate while cell fragments, of e.g. lysed cells, and other smaller components, such as salts, remain in a supernatant.
   c) (optionally) discarding said supernatant
45. The method for supervised training of a machine learning model according to item 44, wherein the lung maturity data is derived from measurements of the precipitate, such as dry transmission FTIR measurements.
46. The method for supervised training of a machine learning model according to any of items 35-45, wherein the GAS data, is derived from, such as comprises or consists of, spectroscopy data, such as mid-infrared spectroscopy data.
47. The method for supervised training of a machine learning model according to any of items 35-46, wherein the GAS data is derived from spectroscopy data in the spectrum between 900-3400 cm⁻¹, such as between 900-1800 cm⁻¹ and between 2800-3400 cm⁻¹.
48. The method for supervised training of a machine learning model according to any of items 35-47, wherein the GAS data is derived from, such as comprises or consists of, one or more absorption and/or transmission spectra.
49. The method for supervised training of a machine learning model according to any of items 35-48, wherein the GAS data is derived from spectroscopy data obtained by spectroscopically analysis of GAS sample(s).
50. The method for supervised training of a machine learning model according to any of items 35-49, wherein the GAS sample(s) is substantially dry during the analysis.
51. The method for supervised training of a machine learning model according to any of items 35-50, wherein the GAS sample is pretreated, prior to spectroscopically analysis.
52. The method for supervised training of a machine learning model according to item 51, wherein the pretreatment comprises or consists of centrifugation for formation of a precipitate, and discarding the supernatant.
53. The method for supervised training of a machine learning model according to any of items 35-52, wherein the GAS sample has been collected, from the infant, by a feeding tube in combination with means of displacing GAS through said feeding tube, such as a syringe, or a suction catheter.
54. The method for supervised training of a machine learning model according to any of items 35-53, wherein the GAS data is derived by application of a mathematical operation to the spectroscopy data.
55. The method for supervised training of a machine learning model according to item 54, wherein the mathematical operation comprises or consists of a 1^{st} order derivative.
56. The method for supervised training of a machine learning model according to any of items 54-55, wherein the mathematical operation comprises or consists of a baseline correction algorithm, such as the Savitzky-Golay algorithm.
57. The method for supervised training of a machine learning model according to any of items 54-56, wherein the mathematical operation comprises or consists of selecting predetermined wavenumbers of the spectrum.
58. The method for supervised training of a machine learning model according to item 57, wherein the predetermined wavenumbers show a statistical significant difference between infants that develop BPD and infants that do not develop BPD.
59. The method for supervised training of a machine learning model according to item 58, wherein the statistical significant difference is based on a statistical test, such as the paired Cox-Wilcoxon test, with a two-tailed p-value <0.05.
60. The method for supervised training of a machine learning model according to any of items 54-59, wherein the mathematical operation comprises or consists of a partial least square analysis.
61. The method for supervised training of a machine learning model according to any of items 35-60, wherein the GAS data is obtained by a process comprising:
   a) (non-invasively) obtaining the GAS sample;
   b) (optionally) storing the GAS sample;
   c) pretreating the GAS sample; and
   d) obtaining spectroscopy data by analysing the GAS sample, by spectrometry, such as mid-infrared spectrometry.
   e) (optional) applying one or more mathematical operations to the spectroscopy data
62. The method for supervised training of a machine learning model according to any of item 35-61, wherein the outcome data comprises or consists of information related to if the infants had, or developed, BPD, such as requiring supplemental oxygen at postnatal day 28.
63. The method for supervised training of a machine learning model according to any of items 35-62, wherein BPD is defined as requirement of supplemental oxygen support at a specific number of days after birth, such as at postnatal day 28.
64. The method for supervised training of a machine learning model according to any of items 35-63, wherein the trained model is a supervised model or a supervised and unsupervised trained model.
65. The method for supervised training of a machine learning model according to any of items 35-64, wherein the trained model is selected from the list including a support vector machine (SVM), a regression model, an artificial neural network, a decision tree, a genetic algorithm, a Bayesian network, or a combination thereof.
66. The method for supervised training of a machine learning model according to any of items 35-65, wherein the prediction comprises or consists of a percentage risk of the infant developing BPD.
67. The method for supervised training of a machine learning model according to any of items 35-66, wherein the sensitivity of the prediction is at least 70%.
68. The method for supervised training of a machine learning model according to any of items 35-67, wherein the specificity of the prediction is at least 70%.
69. A machine learning model for predicting, early after birth, if a subject suffers from, or will develop, bronchopulmonary dysplasia (BPD), wherein the machine learning model has been trained according to any of items 35-68.
70. Use of a machine learning model according to item 69.
71. A system for predicting if an infant, early after birth, will develop BPD, the system comprising
   a) a memory, and
   b) a processing unit that is configured to carry out the method of any of items 1-68.
72. The system according to item 71, further comprising at least one spectrometry unit for obtaining spectrometry data, such as a FTIR spectrometer.
73. The system according to any one of items 71-72, wherein the system is portable and/or a bedside system.

1. A computer-implemented method for predicting risk of an infant developing bronchopulmonary dysplasia (BPD), the method comprising the steps of:
a) obtaining a dataset, of the infant, comprising:
clinical data;
lung maturity data; and
gastric aspirate (GAS) data;
b) analysing said dataset, thereby obtaining an analysed data result; and
c) based on said analysed data result predicting the risk of the infant developing BPD.

2. The computer-implemented method according to claim 1, wherein the dataset consists of data obtained within 48 hours after birth, preferably within 36 hours after birth.

3. The computer-implemented method according to any one of the preceding claims, wherein the clinical data consists of birth weight and gestational age.

4. The computer-implemented method according to any one of the preceding claims, wherein the lung maturity data is derived from measurement data of a bodily fluid sample, comprising GAS, pharyngeal secretion and/or amniotic fluid and/or wherein the lung maturity data is an indicator of whether the infant has been given surfactant treatment or not.

5. The computer-implemented method according to any one of the previous claims, wherein the GAS data is derived from measurements of a GAS sample, such as from measurements data.

6. The computer-implemented method according to claim 5, wherein the GAS data is derived from spectroscopy measurements of the GAS sample, such as from spectroscopy data.

7. The computer-implemented method according to claim 6, wherein the GAS data is derived from spectroscopy data in the spectrum between 900-3400 cm⁻¹, such as between 900-1800 cm⁻¹ and between 2800-3400 cm⁻¹.

8. The computer-implemented method according to any one of claims 6-7, wherein the GAS data is derived from a number of predetermined wavenumbers of the spectroscopy data.

9. The computer-implemented method according to claim 8, wherein the predetermined wavenumbers are selected such that they show a statistical significant difference between infants that develop BPD and infants that do not develop BPD.

10. The computer-implemented method according to any one of claims 8-9, wherein the GAS data is derived from between 10-50 predetermined wavenumbers of the spectroscopy data, such as wherein the spectroscopy data comprises at least 500 wavenumbers.

11. The computer-implemented method according to any of claims 5-10, wherein the GAS data is derived by application of a mathematical operation to the measurement data.

12. The computer-implemented method according to claim 11, wherein the mathematical operation comprises or consists of a 1^{st} order derivative.

13. The computer-implemented method according to any one of claims 11-12, wherein the mathematical operation comprises or consists of a baseline correction algorithm, such as the Savitzky-Golay algorithm.

14. The computer-implemented method according to any one of claims 11-13, wherein the mathematical operation comprises or consists of a partial least square analysis.

15. The computer-implemented method according to any one of claims 5-14, wherein the GAS sample is substantially dry during the measurements.

16. The computer-implemented method according to any one of claims 5-15, wherein the GAS sample is pretreated, prior to the measurements.

17. The computer-implemented method according to claim 16, wherein the pretreatment comprises or consists of centrifugation for formation of a precipitate, and discarding the supernatant.

18. The computer-implemented method according to any one of claims 16-17, wherein the pretreatment comprises:
a) lysing cells present in the GAS sample, such as by mixing with freshwater;
b) centrifugation of the lysed GAS sample, at a rotational centrifugal force (RCF) and time selected such that LBs of the bodily fluid sample forms a precipitate while cell fragments, of e.g. lysed cells, and other smaller components, such as salts, remain in a supernatant;
c) discarding said supernatant;
d) (optional) drying of the precipitate.

19. The computer-implemented method according to any of claims 5-18, wherein the GAS data is obtained by a process comprising:
a. pretreating the GAS sample; and
b. obtaining measurement data, such as spectroscopy data, by measuring the pretreated GAS sample, such as a precipitate by FTIR spectrometry;
c. applying one or more mathematical operations to the spectroscopy data.

20. The computer-implemented method according to any one of the preceding claims, wherein BPD is defined as a requirement of supplemental oxygen support at a specific number of days after birth, preferably 28 days.

21. The computer-implemented method according to any one of the preceding claims, wherein the prediction comprises or consists of a percentage risk of the infant developing BPD.

22. The computer-implemented method according to any one of the preceding claims, wherein the analysed data result is obtained by analysing the dataset by a trained machine learning model.

23. The computer-implemented method according to claim 22, wherein the trained model is a support vector machine (SVM), trained by supervised learning.

24. A method for supervised training of a machine learning model for predicting, early after birth, if a subject suffers from, or will develop, bronchopulmonary dysplasia (BPD), the method comprising:
a) obtaining a dataset, comprising information of a number of infants shortly after birth, comprising
clinical data, consisting of birth weight and gestational age;
lung maturity data, consisting of an indication of whether the infant has been given surfactant treatment or not; and
gastric aspirate (GAS) data;
b) obtaining outcome data comprising or consisting of information related to if the infants had, or developed, BPD;
c) training a machine learning model, by supervised training, based on the dataset and the outcome data of the infants, to predict, early after birth, if a subject suffers from and/or will develop BPD.

25. The method according to claim 24, wherein the machine learning model is trained to carry out the method of any one of claims 1-23.

26. A system for predicting if an infant, early after birth, will develop BPD, the system comprising
a) a memory;
b) at least one spectrometry unit configured for obtaining spectrometry data, such as an FTIR spectrometer;
c) a processing unit that is configured to carry out the method of any one of claims 1-25.

27. The system according to claim 26, wherein the system is portable and/or a bedside system.
